SEQUENCE LISTING (2 sequences)

SEQ ID NO: 1 - protein, 590 aa, Mammary Tumor Virus
GVSGSKGQKLFVSVLQRLLSERGLHVKESSAIEFYQFLIKVSPWFPEEGGLNLQDWKRVGREMKRYAAEHGTDSIPKQAYPIWLQLREILTEQSDLVLLSAEAKSVTEEELEEGLTGLLSTSSQEKTYGTRGTAYAEIDTEVDKLSEHIYDEPYEEKEKADKNEEKDHVRKIKKVVQRKENSEGKRKEKDSKAFLATDWNDDDLSPEDWDDLEEQAAHYHDDDELILPVKRKVVKKKPQALRRKPLPPVGFAGAMAEAREKGDLTFTFPVVFMGESDEDDTPVWEPLPLKTLKELQSAVRTMGPSAPYTLQVVDMVASQWLTPSDWHQTARATLSPGDYVLWRTEYEEKSKEMVQKAAGKRKGKVSLDMLLGTGQFLSPSSQIKLSKDVLKDVTTNAVLAWRAIPPPGVKKTVLAGLKQGNEESYETFISRLEEAVYRMMPRGEGSDILIKQLAWENANSLCQDLIRPIRKTGTIQDYIRACLDASPAVVQGMAYAAAMRGQKYSTFVKQTYGGGKGGQGAEGPVCFSCGKTGHIRKDCKDEKGSKRAPPGLCPRCKKGYHWKSECKSKFDKDGNPLPPLETNAENSKNL

SEQ ID NO: 2 - protein, 98 aa, Mammary Tumor Virus
GVSGSKGQKLFVSVLQRLLSERGLHVKESSAIEFYQFLIKVSPWFPEEGGLNLQDWKRVGREMKRYAAEHGTDSIPKQAYPIWLQLREILTEQSDLVL

(19) United States
(12) Patent Application Publication
(10) Pub. No.: US 2026/0325497 A1
(43) Pub. Date: Oct. 22, 2026

(54) MAMMARY TUMOR VIRUS SUPPRESSION

(71) Applicant: VIRAGO VAX INC., NEW YORK, NY (US)

(72) Inventor: SOPHIE BARTSICH, NEW YORK, NY (US)

(73) Assignee: VIRAGO VAX INC., NEW YORK, NY (US)

(21) Appl. No.: 18/664,913

(22) Filed: May 15, 2024

Related U.S. Application Data
(63) Continuation of application No. 16/920,089, filed on Jul. 2, 2020, now Pat. No. 12,016,844.
Application No. 16/920,089 is a continuation-in-part of application No. 16/490,295, filed on Aug. 30, 2019, now Pat. No. 10,891,816.
(60) Provisional application No. 62/934,858, filed on Nov. 13, 2019.

(51) Int. Cl. A61K 31/427; A61K 31/4418; A61K 39/21; C07K 14/005; C12N 7/00; G01N 33/57515

(52) U.S. Cl. CPC A61K 31/427; A61K 31/4418; A61K 39/21; C07K 14/005; C12N 7/00; G01N 33/57515; C12N 2740/12022; C12N 2740/12034

(57) ABSTRACT

Disclosed are methods of treating a patient exposed to MTV. Also disclosed is a purified cDNA encoding an MTV transition protein peptide chain. Also disclosed is a vaccine containing an MTV transition protein. A further version of the invention is a vaccine comprising MTV polypeptides coupled to a carrier protein. MTV may be treated by providing an MTV vaccine with an MTV transition protein; and, administering said vaccine. In further instances, the MTV vaccine is administered with an antiretroviral medication.

### APPLICATION CROSS-REFERENCE

The instant application is a continuation application of U.S. patent application Ser. No. 16/920,089, filed on Jul. 2, 2020, which application is a Continuation-in-Part of U.S. application Ser. No. 16/460,295 filed on 2 Jul. 2019, which also relates to PCT/US19/40361 filed on 2 Jul. 2019, and which further relates to U.S. Provisional Patent Application Ser. No. 62/934,858 filed on 13 Nov. 2019. The disclosures of each of the foregoing applications is hereby incorporated by reference herein in their entireties.

### SUBMISSION OF SEQUENCE LISTING XML FILE

The instant application contains a Sequence Listing which has been submitted electronically in XML format and is hereby incorporated by reference in its entirety. Said XML file, created on 19 May 2025, is named 1133-0002-1-1_SL_CORRECTED.xml and is 3,413 bytes in size. The content of the XML file incorporated herein by reference is a computer readable form (CRF) of three sequences:
SEQ. 1 Illustrates an example Pr75gag polyprotein sequence.
SEQ. 2 Illustrates an example transitional protein p10 sequence.
SEQ. 3 Illustrates an artificial cDNA sequence encoding a modified p10 transitional protein.

### BACKGROUND

### Technical Field

Embodiments of the invention generally fall into the category of suppression of viral replication. In some embodiments a protease inhibitor is used to control viral replication to prevent recurrence or metastasis in patients with invasive viral-mediated cancers. In other embodiments the protease inhibitor is combined with a cDNA produced peptide.

### Discussion of Art

The etiology of breast cancer is likely multi-factorial. Genetics are known to play a role (BRCA for example), as do diet, and hormones (Dudley, 2016). The mammary tumor virus (MTV) is found across a broad range of species, mammals in particular. The virus is implicated as a general tumor-forming virus, suggesting that it may well be a wide-ranging zoonotic tumor virus with deep penetration throughout our shared environment.

The Mouse Mammary Tumor Virus (MMTV) was first described by Bittner in 1936, in the context of its being an exogenous infectious agent that was transmitted from mother mouse to baby mouse through breastmilk. Weiss later categorized it as a B type retrovirus, comparable to HIV, but carried in far smaller concentrations (1996). As a B type retrovirus, MTV belongs to a group globally recognized as oncogenic. As such, it acts through insertional mutagenesis, ultimately producing a tumor in its target tissue.

MMTV strains vary according to species of mouse, which itself varies by geography and historical custom. The observance of the parallel trends in MMTV expression and human breast cancer led to a probable associative link between the two. Areas that have higher concentrations of rodents and have had them for a longer period of time show increased virulence and increased rates of cancer. In fact, the incidence and manifestation of breast cancer in humans can be directly correlated to the species of mouse endemic to a given area. Regions where mouse populations are generally low, like East Asia, have lower overall breast cancer rates, and those populations have tumors that exhibit very low virus positivity (Khan, 2008; San, 2017). The most virulent strains of MMTV lie in the Middle East, an area with a higher rodent population and where breast cancer onset and severity are more pronounced than elsewhere (Chouchane, 2013). In the United States and Mexico, where mice are ubiquitous, tumor samples test positive for the MMTV virus (Cedro, 2014) and can be linked to the “house mouse,” mus domesticus (Stewart, 2000).

A hormonally-activated and regulated retrovirus, the genetic code of MTV is fully deciphered for variants found in some mammalian species. The genetic code of the deciphered variants share a common genetic architecture with many other retroviruses with major expression groups comprising: long terminal repeats (LTR); group antigen polyproteins (GAG); reverse transcriptases and polymerases (POL); and, envelope proteins (ENV). FIG. 1 provides an illustration of this generalized architectural structure for MTV. The proteins that compose the GAG group generally form the viral core structure, RNA genome binding proteins, and are the major proteins comprising the nucleoprotein core. The GAG group may also encode viral matrix, capsid, and nucleoproteins. The POL reverse transcriptase is the essential enzyme that carries out the reverse transcription process that takes the viral RNA genome to a double-stranded DNA pre-integrated form. The ENV group encodes for host cell transmembrane and surface-receptor subunits, and regulatory subunits found on the endoplasmic reticulum of the host cell in addition to the proteins that form the viral envelope.

The MMTV virus, while endemic to mice, appears to be acquired within the species itself. An antigenic component, the gp52 envelope protein can reliably be found in the blood and reproductive organs of infected mice (Arthur, 1978), as can antibodies to envelope proteins such as gp52. The virus is passed through breast milk, urine, feces, and/or saliva, and it travels liberally until it finds hormone-dependent tissues to infect, with breast being the preferred target. Once inside a cell, the virus randomly inserts into a series of integration sites (Faschinger, 2008), ultimately meeting an oncogene and producing the necessary frameshift to cause a cancer-producing mutation (Moore, 1987).

Due to the apparent connection between MMTV exposure and breast cancer in mice, coupled with the above mouse population-human breast cancer prevalence observations, many efforts have been made to find a human correlate to the virus, the Human Mammary Tumor Virus (HMTV), and its subsequent role in breast cancer in women. A human corollary found to be 85-95% homologous to MMTV was described by Melana and Holland in 2001. This HMTV was identified in human breast cancer specimens, first through identification of homologous MMTV envelope proteins (Wang, 1995) and later with the discovery of proviral units specific to human subjects (Liu, 2001).

While the prevalence of HMTV in breast cancer tissue has varied widely across studies, there is evidence that it is consistently well-represented in certain types of cancers, namely ductal carcinoma in situ (DCIS; Mazzanti, 2011). Even in subjects with significant disease, the virus may be somewhat elusive, owing to the fact that it is present in low concentrations (Bindra, 2007), and serves as a relatively remote precursor to oncogenesis (Nartey, 2017). Viral acquisition predates tumor formation by one to eleven years (Lawson, 2017), with the virus itself possibly becoming less virulent after initial mutagenesis. Ultimately, viral particles can be detected in tumor tissue and breast milk (Nartey, 2014), demonstrating a predilection for that organ system. The affinity for breast tissue includes male subjects as well, in whom particles have been isolated from benign cases of gynecomastia (Ford, 2004).

HMTV shares homology with MMTV in both form and action and the two share similar genetic sequences. As used herein the term “homolog” and its derivatives are, unless specified with a percentage, used as commonly understood by practitioners in the field of biochemistry in that proteins sharing similar behavioral properties, structural motifs, or performing similar functions with conserved domains across a family are said to be “homologous” to each other. Likewise, sequence data for DNA or RNA may be more or less “homologous” in that shared identities across species are said to be “homologous.” When used as a percentage, homologous is interpreted to mean a comparison of sequences (DNA, RNA, peptide) with sequences above a certain percentage similarity or bearing certain conserved structural motifs at key positions said to be “homologous” with other sequences.

Human breast tumors demonstrate evidence of envelope protein units, as well as equivalent RNA segments known to originate from MMTV (Axel, 1962). The HMTV virus inserts similarly into the genome at random sites, ultimately inciting comparable mutations by activating various oncogenes (Callahan, 2012). Representatives of the envelope protein can be localized to the host cell membrane, but are more prominent in cellular cytoplasm (Tomana, 1981).

In general, transcription processes read through an MTV genome and mRNA is polyadenylated and processed using signals in transcribed regions from the 3′ LTR at the end of the transcribed RNA. The full-length message can be spliced to lead to production of envelope proteins (or other proteins depending upon retroviral class). Unspliced full-length mRNA can give rise to GAG-POL precursor polyproteins. Alternatively, GAG and POL proteins, and other MTV proteins, or protein subunits may first arise from translated mRNA polypeptide precursor molecules. Multiple precursors are possible and may contain multiple proteins or protein subunits. A viral protease cleaves the precursor into multiple subunits with varying functions. An ENV protein complex, for example, may be translated from mRNA segments into multiple precursors which are then cleaved by endogenous proteases to yield multiple subunits that assemble into a mature surface glycoprotein.

Translated proteins assemble a retroviral particle at the host cell surface. Full-length genomic unspliced mRNA (containing a packaging signal termed Psi) is bound by GAG-derived proteins and incorporated into the budding particle. As a B-type retrovirus the general structure of a mature MTV particle is characterized by prominent surface protein “spikes” and a dense acentric nucleocapsid.

The discovery of Human Endogenous Retroviruses (HERV) DNA sequences embedded in the genome led to the hypothesis that many retroviruses, or genetic elements derived from retroviruses, are now endogenous to humans; the result of many generations of people being repeatedly exposed to the agents and eventually integrating them into their DNA. The HERV equivalent to MTV has been found in human subjects (Salmon, 2014). Some researchers hypothesize that HMTV is purely endogenous, and that efforts at curtailing its oncogenic potential should be directed at genetic manipulation rather than immunization against an exogenous entity (Yang, et al., 1987).

MTV behaves in a fashion more consistent as an exogenous particle (Ford, 2004; Melana, 2007). The fact that the virus is acquired up to ten years prior to disease onset in view of the fact that the mean age of onset of breast cancer is over 50 leads the argument for an endogenous factor to fall short. Furthermore, the measured immunogenicity against MTV is consistent with that of an acquired infection, and is maximal during disease latency (Black, 1976).

In the setting of MTV and its probable role in breast cancer, there is strong evidence in support of a combinatorial effect of MTV with multiple oncogenic viruses acting in conjunction and potentiating each other during tumorigenesis (Corbex, 2014; Glenn, 2012; Lawson, 2018; Al Moustafa, 2016). Taken as a whole, the data on MTV suggests that there are in fact endogenous and exogenous versions of the virus, many with little clinical relevance to immediate disease. It is also probable that the HERVs previously described represent remnants of prior integrations, and that newer incidences of MTV infection ultimately lead to cancer onset.

The retrovirus replication cycle is characterized by conversion of the single-strand RNA viral genome into double-strand proviral DNA by the multiple enzymatic activities of the virus-associated reverse transcriptase. Integration of MMTV proviral DNA into the genome of host cells is required for the expression of viral proteins. In both infected mouse mammary glands as wells as heterologous cells MMTV proviral DNA is integrated into a large number of apparently random sites. Integration of MMTV proviruses containing transcriptionally active long terminal repeats (LTR) near some cellular genes (proto-oncogenes) involved with oncogenesis can result in over-expression of these genes resulting in cellular transformation and clonal expansion of tumor cells. Thus, the long latency of MMTV-induced carcinogenesis is explained in part by the necessity for proviruses to integrate into oncogenetic sites.

Efforts at measuring MTV antibodies have had varying success. Antibodies to some envelope proteins have been isolated, although sparingly, as they seem to wane with disease progression as previously mentioned. The MTV virus itself also appears to have evolved mechanisms that blunt the host response to infection (Dudley, 2016). Previous diagnostic tests for MTV in humans have been limited to localizing the envelope protein in the host, as opposed to finding the more prevalent and longer lasting intracellular elements. (Holland, 1995) A further example of a similar attempt may be found in Garry (U.S. Pat. No. 6,670,466) the teachings of which are hereby incorporated by reference in their entirety.

While mice are ubiquitous and are known to be present in both homes and food storage facilities, MTV appears to require more intimate contact for effective transmission. Various efforts to detect MTV in other species have shown that correlates are found in primates, feline and canine species, in addition to the well-known mouse model (Szabo, 2005). Cross-species transmission is possible, as evidenced by the acquisition of MMTV in lab personnel who performed frequent handling of infected mice (Dion, 1986).

MTV appears to require repeated and frequent exposure for effective transmission. To that end, domesticated pets have become the prime targets for study, and the prime suspects as vectors. MTV particles are found in the non-cancer cells of house pets, with sequences similar to those of infected mice (Hsu, 2010). Pet-human interaction being frequent and extensive, studies then proceeded to identify the most common mode of transmission between them. Given that MTV has been detected in various body fluids, saliva was next to be evaluated for its virulent potential.

In 2015, Mazzanti identified MMTV particles in human saliva, suggesting that to be one mode of transmission between humans. Extrapolating from that data, it is likely that the house pets in whom MTV variants are found also have the virus present in their saliva, and pet saliva itself is ubiquitous in human homes. Dog ownership in particular has been isolated as an independent risk factor for breast cancer in women (Laumbacher, 2006), a fact anecdotally replicated on a daily basis in oncologic surgery practices in the United States.

Finally, given the above putative associations between MTV and cancer there have been past attempts at creating an MTV vaccine. However, the prior attempts at generating an MTV vaccine have used ENV proteins or protein parts (e.g., Garry) and failed to raise the required immune response. Thus, there is a need for an MTV vaccine capable of inducing immunity in humans and in human-associated animals. Further, as the oncogenic properties of exogenous MTV are only recently recognized, there is a need to develop treatments effective at lowering viral particle load and/or activity.

### BRIEF SUMMARY OF THE INVENTION

An embodiment of the invention is a method of reducing breast cancer recurrence via prevention of MTV transcription in breast tissue via administration of a protease inhibitor. Other compositions contemplated as part of various embodiments of the instant invention provide a means for stabilizing or reducing the amount of virus present in a human or other animal.

In another embodiment of the invention a patient is first tested for the presence of MTV. Upon discovery of MTV the patient is then administered an antiretroviral medication. In some embodiments the antiretroviral medication is a protease inhibitor. In other embodiments the antiretroviral treatment may be one or more selected from the group of: nucleoside analogue reverse transcriptase inhibitors, nucleotide analogue reverse transcriptase inhibitors, non-nucleoside reverse transcriptase inhibitors.

In still other embodiments the antiretroviral medication, such as a protease inhibitor, is administered to a subject at a first dosage level, corresponding to a treatment level, and then altered to a second or more dosage level corresponding to a maintenance level. In still other embodiments the test for MTV presence is an antibody titer test. In still other embodiments the antibody test relies on antibodies raised in response to at least one of: GP52, a transition protein, and p14. In still other embodiments the transition protein is p10.

In another embodiment a patient with a breast cancer diagnosis, or a patient with breast cancer risk factors is treated with an antiretroviral medication. In still other embodiments this treatment is combined with the administration of MTV transition proteins or transition protein peptides.

In still another embodiment a cDNA encoding an MTV transition protein is provided. The protein may be myristoylated and, likewise, the cDNA may encode for this modification. Also provided are cDNA molecules encoding MTV transition protein peptide chains of greater or lesser length with or without a myristol. The transition protein or transition protein peptide chains may then be incorporated as part of an MTV vaccine or treatment regimen administered to a patient for control of MTV particle count or viral activity.

### BRIEF DESCRIPTION OF THE DRAWINGS

Features and advantages of embodiments of the present invention will become apparent on reading the detailed description below with reference to the drawings, which are illustrative but non-limiting, wherein:

FIG. 1 Illustrates the general generic genomic structure of a generic MTV.

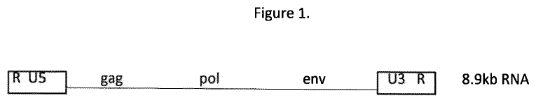

FIG. 2 Illustrates a schematic representation of Pr75gag cleavage products.

FIG. 3 Illustrates a schematic of an MTV virus.

### DETAILED DESCRIPTION OF THE INVENTION

Although described herein as singular genetic elements, each class of ENV, GAG, POL, and LTR, may encode for multiple proteins, subunits, glycosylation sites, or signaling factors. Vaccines also include a variety of ingredients besides classifying antigens and can include: stabilizers, adjuvants, antibiotics, and preservatives. Those skilled in the art of pharmaceutical preparation are able to select appropriate additional ingredients in the course of standard practice. In this instance the term “vaccine” is used as ordinarily understood and may also contain a secondary descriptor describing the antigenic component eliciting the desired immune response along with the associated ingredients tailored to the desired effect. It is further understood that a vaccine may be all or part of a “pharmaceutical preparation” as herein defined and may incorporate a combination of natural and synthetic components. In certain instances it is recognized in the art that bringing together two natural components, a viral piece and an adjuvant, give rise to a composition with properties greater than the sum of its parts. (i.e., conferring a greater immune response, providing stabilization of antigens, etc.) It is those enhanced, non-naturally occurring properties to which the term “vaccine” further contemplates.

There are many types of vaccines, categorized by the antigen used in their preparation. Their formulations affect how they are used, how they are stored, and how they are administered. There are at least four main types of vaccine: Live Attenuated Virus (“LAV”), Inactivated (killed antigen), Subunit (purified antigen), and Toxoid (inactivated toxins). Vaccines may be monovalent or polyvalent. A monovalent vaccine contains a single strain of a single antigen (e.g. Measles vaccine), whereas a polyvalent vaccine contains two or more strains/serotypes of the same antigen (e.g. OPV). Some of the above antigens may be combined in a single injection that can prevent different diseases or that protect against multiple strains of infectious agents causing the same disease (e.g. combination vaccine DPT combining diphtheria, pertussis, and tetanus antigens). Thus, combination vaccines can be useful to overcome the logistic constraints of multiple injections and accommodate for a patient's fear of needles and pain. As used in this application, the term “polyvalent vaccine” refers to multiple antigen types that may derive from a single or multiple disease vectors whereas a “combination vaccine” refers to a vaccine with multiple antigens targeting at least two or more diseases. Thus, a “polyvalent vaccine” may have multiple antigens but only provide immunity to a single disease whereas a “combination vaccine” may contain a single type of antigen from two or more disease vectors and give rise to an immunity for those two or more diseases. Also, as used herein the term “patient” reflects a human or animal subject to whom a medical treatment is provided either for modification of a disease state or as a prophylactic preventative of disease.

Available since the 1950s, live attenuated vaccines (LAV) are derived from disease—causing pathogens (virus or bacteria) that have been weakened under laboratory conditions. They will grow in a vaccinated individual, but because they are weak, they will cause no or very mild disease. Inactivated vaccines are made from microorganisms (viruses, bacteria, other) that have been killed through physical or chemical processes. These killed organisms cannot cause disease.

Subunit vaccines, like inactivated whole-cell vaccines do not contain live components of the pathogen. They differ from inactivated whole-cell vaccines, by containing only the antigenic parts of the pathogen. Protein based subunit vaccines present an antigen to the immune system without viral particles, using a specific, isolated protein of the pathogen. Some bacteria when infecting humans are often protected by a polysaccharide (sugar) capsule that helps the organism evade the human defense systems especially in infants and young children. Polysaccharide vaccines create a response against the molecules in the pathogen's capsule. Conjugate subunit vaccines also create a response against the molecules in the pathogen's capsule. In comparison to plain polysaccharide vaccines, they benefit from a technology that binds the polysaccharide to a carrier protein that can induce a long-term protective response.

Toxoid vaccines are based on the toxin produced by certain bacteria (e.g. tetanus or diphtheria). The toxin invades the bloodstream and is largely responsible for the symptoms of the disease. A protein-based toxin is rendered harmless (toxoid) and used as the antigen in the vaccine to elicit immunity. To increase the immune response, the toxoid is often adsorbed to aluminum or calcium salts, which serve as adjuvants.

Thus, a live vaccine can have different associated ingredients than a subunit-based vaccine. Both are vaccines and raise an immune response in patients but with different antigens. Or, in the alternative, two live vaccines may have the same antigen but varied ingredients (e.g., in response to administration requirements). Or, in other embodiments, the vaccine may have the same antigen (e.g., an ENV protein or protein subunit) that is derived from one or more sources (e.g., an ENV protein from a human and one from a cat). In other embodiments, two or more antigenic components may be combined to increase immune system response greater than an individual antigen alone (e.g., an ENV protein combined with a protein precursor). Additionally, antigens for MTV may be combined with other antigens for other diseases into a mono or polyvalent combination vaccine.

The term “antigens” and derivatives are defined as the components typically derived from the structure of disease-causing organisms, which are recognized as “foreign” by the immune system and trigger a protective immune response to the vaccine. Importantly, the antigen component does not have to be made by the disease-causing organism in order to be used. For example, viral proteins or protein precursors may be manufactured and purified using bacterial, fungal, cell free, or other vector-based systems in bulk and then purified for use in a vaccine serum or other pharmaceutical preparation.

Vaccines may be administered through multiple routes at least including: intramuscular injection, subcutaneous injection, intradermal injection, orally, or through intranasal sprays. It is understood that each route of administration may require its own mix of antigens and associated ingredients.

In this application synthetic proteins and derivatives are prefixed with an “syn” such as synMTV or synENV, synPOL, etc. to differentiate from naturally occurring versions. For example, an synENV protein may be derived from a genetic sequence identical to or modified from (e.g., introns removed, different starter code and reading frame, different promoters etc.) a naturally occurring sequence but produced in an alternative system of protein expression (e.g., in yeast or e. coli, etc.). Likewise, the amino acid sequences, and underlying genetics, for each protein may vary across viral sub-species found in different mammalian species. synProteins or synProtein subunits (such as those produced by a protein cleavage process, those produced by restriction processes, or those produced by artificial amino-acid peptide chain construction, amino acid printing, or other artificial systems or protein or protein product expression) may be derived in whole or in part from single or multiple viral sub-types (e.g., human sequence synProtein combined with a homologous canine synProtein to form a polyvalent vaccine protective against both HMTV and canine MTV virus particles).

In an example embodiment, an engineered vaccine is created by synthesizing and purifying an EP3-p14-p10 peptide chain. The peptides, in haptan formulation, are coupled to keyhole limpet hemocyanin (KLH) and extensively dialyzed. Moles of conjugated peptide/mole of KLH are determined by amino acid analysis and range between 700 and 1,000. Positive and negative controls consist of gp52, purified from C3H-MMTV and a scrambled peptide sequence coupled to KLH respectively. Still other embodiments may employ different carrier proteins including at least: Concholepas concholepas hemocyanin, bovine serum albumin (BSA), cationized BSA, or ovalbumin, and any other carrier proteins. In still another embodiment, multiple antigens, for instance from other viral-mediated cancers or tumor-associated antigens, or for other diseases which may be vaccinated against, may also be attached to one or more carrier proteins and combined to form a polyvalent combination vaccine. In practice, the vaccine is administered to patients either as a prophylactic preventative or, in the case of patients with a known MTV exposure as a means of inducing or bolstering an immune response.

The MTV envelope (ENV) class of proteins is known to have conserved domain elements across variants of the virus detected across multiple species. In particular, the ENV proteins may comprise those that are signaling peptides, those that are specific to the outer membrane of the virus particle, those specific to the endoplasmic reticulum, and those that are trans-membrane anchors. The polymerase (POL) protein class may comprise both a polymerase and/or a reverse transcriptase and an associated signaling factor. The group antigen (GAG) proteins form the viral core structure, RNA genome binding proteins, and are the major proteins comprising the nucleoprotein core particle. The POL and GAG group proteins are likewise thought to have conserved cross-species elements.

Without subscribing or limiting to any specific theory or practice, variants within and between the above genetic motifs may account for viral traits such as specificity, genetic integration location, surface protein structures, species specificity, etc. Thus, by altering combinations one may target a vaccine to protect against an MTV exposure in various species. This may be accomplished by either deriving viral proteins from the target species of interest or utilizing synProteins or synProtein subunits derived from species-specific sequence data. A target species may be one whose MTV viral protein sequences, although homologous, differ enough from the primary MTV antigen in a vaccine that a separate antigen is required to raise or enhance an immune response either in the species of interest or to increase an immune response, for example in a human, in reaction to an exposure of an MTV variant from the target species. For example, a human MTV vaccine with a transition protein may include additional MTV polypeptide antigens whose sequences are derived from human companion species such as a dog, cat, ferret, etc. Thus, while the vaccine may also possibly be administered to the human-companion animal to reduce viral load in the animal (and hence exposure to the human), the additional MTV component also strengthens the immune response of the human to the target species variant of MTV.

Accordingly, another embodiment of the invention comprises an MTV vaccine derived from attenuated or inactivated MTV particles derived from either single or multiple species. Other embodiment vaccines are derived from synProteins and synProtein subunits of the ENV, POL, and GAG protein classes. An additional embodiment is composed of synProteins and/or synProtein subunits derived from cross-species conserved sequences. For example, some portions of the sequence of the gp52 ENV protein are known to be conserved across MTV variants. Thus, a synProtein subunit based on the conserved sequence could be used in a vaccine to induce immunity to MTV in multiple species, including human-associated animals such as dogs, cats, and other human-companion animals or increase the immune response in a human to MTV variants derived from the animals.

As used herein the term “derived from” is used as ordinarily understood: that is, a sequence (DNA, RNA, or peptide) may be found across multiple species with minor variations while still preserving a high sequence identity. Thus, while one can recognize, for example, MTV protein p10 in general, it is the unique mutations, sometimes in the form of single nucleotide polymorphisms (SNPs), that allow one skilled in the art to identify a p10 sequence from a human, compared to another mammal, such as a mouse. In another sense, in addition to identifying sequence sources, “derived from” encompasses and contemplates the source of raw stock materials, binders, supports, carrier proteins, etc. (e.g., KLH is derived from a protein found in keyhole limpets even if commercial embodiments of it are produced in alternative production systems, such as vector-based expression systems).

In still another example embodiment, a purified MTV virus from one or multiple species is combined with 1.2×10⁻² M formalin for a concentration of 1:3,000 formaldehyde. It is then incubated in the dark at 4° C. for 5 days (or room temperature for 72 h) and stored at 4° C. It is diluted to contain 50 ng protein μl⁻¹ and emulsified with an equal volume of complete Freund's adjuvant (CFA) or other pharmaceutically acceptable adjuvant. The preparation is then stored at −70° C. until used. In some embodiments the above serum is combined with one or more additional vaccine sera to form polyvalent, compound, or compound polyvalent vaccines targeted at one or more additional diseases. The resulting pharmaceutical preparation is then administered to a patient for either prophylactic or treatment purposes.

In another example embodiment, MTV virus particles from one or multiple species may be subjected to fragmentation processes designed to break apart the virus into multiple protein and protein pieces (e.g., sonication, vibration, freeze-fracture, etc.). MTV proteins and/or protein subunits may then be collected, purified, and fixed in a vaccine preparation. Particular preparations may include purified ENV, POL, or GAG proteins or protein subunits. In a further embodiment the purified proteins may include p10, p14, and gp52.

As briefly outlined above, RNA viruses in many families and genera express their genomes in ways which involve the synthesis and subsequent cleavage of polypeptide chains known as precursor polyproteins. This stratagem allows the activation of subsets of proteins with different biochemical functions from the same precursor polyprotein. Although the virus-encoded enzymes responsible for processing the polyproteins are structurally diverse, they are all highly specific for their substrates. The resulting processing cascade is a tightly controlled process, which in several cases involves the action of protein cofactors to modulate the activity of the proteinase. Antigens for use in a vaccine can derive from protein precursors.

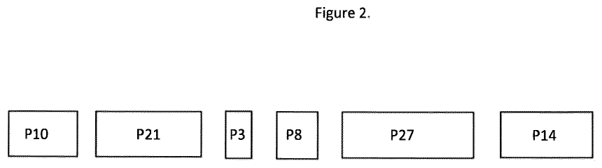

In general, MTV proteins are synthesized with at least two major precursor polyproteins, for MMTV this includes: gPr75env containing gp52 and gp36; and, Pr75gag containing p27, pp20, p14, and p10. SEQ. ID: 01 illustrates an example sequence for a Pr75gag precursor polyprotein, the cleavage products of which are schematically illustrated in FIG. 2. Shown in FIG. 2, for example, p14 is a signal peptide subunit of an ENV protein. p14 can also function in both oncogenic and an anti-oncogenic capacity depending on its phosphorylation status. Also shown in FIG. 2, p10 is part of the same polyprotein precursor as p14 and is also found as a cleavage product thereof. SEQ. ID: 02 illustrates an example sequence of a p10 protein. Those skilled in the art readily recognize that the above sequences are conserved across multiple species without significant deviation. It is a routine skill to find and identify homologous sequences in multiple species and, indeed, homologous sequences for p10 are found in humans, mice, cats, and canines.

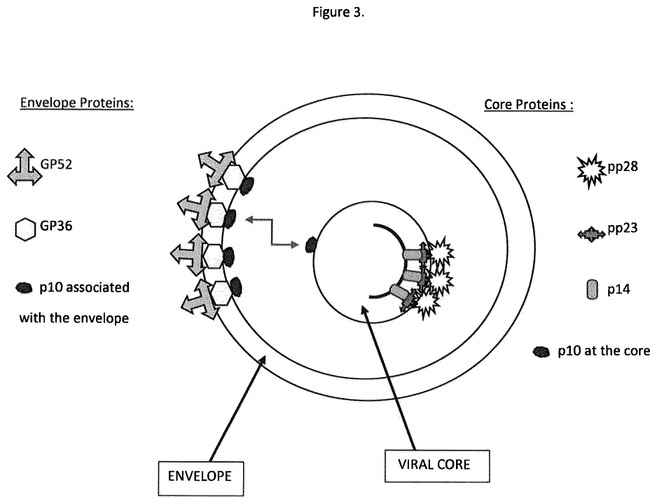

As illustrated in FIG. 3, p10 initially starts at the viral core and then transitions to the envelope membrane of the virus. This core-to-envelope transition protein is believed to be a representative member of a larger class of core-to-envelope proteins. As used herein the term “transition protein” describes a protein, that moves between the viral core and the viral envelope (i.e., from core to envelope or envelope to core). In an embodiment the transition protein may start at the viral core and then move to the viral envelope. The transition protein may thus remain at the underside of the envelope, may transition through the envelope to the exterior, or may span across the viral envelope. A transition protein may also be a protein subunit that forms part of a larger protein. It may be directly produced from viral mRNA, viral DNA, cDNA, or be the result of cleavage from a precursor polyprotein either naturally produced in vivo or synthetically produced (e.g., in a vector-based production system). Thus, an embodiment of the invention includes an MTV transition protein either alone or in combination with further MTV proteins. An MTV transition protein may also be synthetically generated and combined with other antigens or formed as part of a polyvalent, or compound polyvalent vaccine targeting one or multiple diseases and administered to a patient either prophylactically or as a modifier of a disease state.

Thus, in another embodiment an MTV vaccine preparation containing one or more precursor proteins either derived from polyprotein cleavage products or directly created using alternative protein expression means. In particular polyprotein cleavage products, p10, p14, and, alone or in combination with each other, may be combined with at least one ENV, GAG, or POL protein or protein derivative. Still other embodiments may include gp52, gp36, and p28 proteins. These may then be combined with an MTV transition protein, such as p10.

In addition to MTV, other virus species, termed oncoviruses, are possible sources of oncogenesis; these can include at least: Epstein-Barr Virus (EBV) (Ballard, 2015; Lebreque, 1995), human immunodeficiency virus (HIV), human papilloma virus (HPV), herpes simplex virus (HSV), and bovine leukemia virus (BLV) (Baltzell, 2018; Buehring, 2017). It is believed, without subscribing or limiting to a particular theory, and as briefly outlined above, that viruses often work together to potentiate each other and to cause disease in general. Oncogenic viruses in particular can work together to cause disease. Specifically, RNA viruses tend to be unstable but highly virulent, DNA viruses tend to be stable but not as aggressive. DNA and RNA viruses can work together, each building upon the strengths of the other to increase pathogenicity. It is believed that many viruses like HPV and HSV can make other viruses more virulent, and that certain combinations are particularly effective at inducing oncogenesis in patients.

Further, RNA viruses and DNA viruses may have a complimentary effect wherein the high replication and mutation rates of RNA viruses may be stabilized by the less mutagenic DNA viruses, allowing for optimal adaptability without loss of control. The highly active MTV RNA virus, as discussed above, replicates quickly and randomly inserts into the genome, often producing no effect. Genomically-integrated MTV sequences can be found with little to no clinical relevance, and these are often misinterpreted as being endogenous. Once stabilized and directed by a DNA partner, the usually erratic agent can now target its source more effectively and remain consistent long enough to provoke oncogenesis. This is one possible mechanism for HPV/MTV synergy in cancer formation.

Accordingly, embodiments of the invention are directed towards the treatment of breast, prostate, and other viral-mediated cancers or other synergestic diseases in humans and other animals. In some embodiments a protective immunity is formed in patients as a result of vaccination with MTV or synMTV either in whole, in part, or in combinations as part of live, attenuated, or subunit vaccines along with antigenic components of other oncogenic virus particles such as at least: EBV, HIV, HPV, HSV, and BLV. In other embodiments, an MTV vaccine is administered to a patient diagnosed with a viral-mediated cancer with the effect of reducing the malignancy, spread, or recurrence of the viral-mediated cancer.

As noted above, the incidence of breast cancer in humans is increased in areas with increased mouse populations. Also, as above, humans with pets may also experience an increased incidence of breast cancer. Any of the above described vaccines may be altered to target a particular species. For example, a monovalent vaccine targeting a rabbit may use an MTV protein or protein particles purified from rabbit-specific MTV. Likewise, a human targeting vaccine may use ENV protein purified from HMTV strains found in humans. Or, in the alternative, a synthetically derived synProteins or hybrids may be created based upon a target species MTV genetic sequence. In alternative embodiments MTV derived POL or GAG proteins or protein subunits may be used. In an alternative embodiment the vaccine may be composed of peptides derived from one or more conserved sequences found across target species, for example, human, mouse, canine, feline, bovine, and other human-companion animals. Thus, a multi-species MTV vaccine may be created. Additionally, animal vaccination may be used in tandem with human vaccination to decrease the overall incidence of breast cancer and other viral mediated cancers in a human population by decreasing human exposure to MTV transmitted via animals.

Another embodiment of the invention is a polyvalent vaccine comprised of multiple MTV antigen components. In certain embodiments the components may be a mixture of natural and synthetically derived components, for example, a synthetic synENV mixed with a naturally occurring ENV or POL. The polyvalent vaccine may further contain precursor cleavage products such as p10, mp10 (which is a p10 peptide in myristoylated form with or without subsequent conformational changes), or p14. These may further be combined with one or more transition proteins and may be further combined into a combination vaccine or suitable pharmaceutical preparation.

An embodiment of the invention is a vaccine comprising an MTV transition protein administered to a patient. In some embodiments the MTV transition protein is p10. In some embodiments the vaccine further comprises additional antigens to form a combination vaccine. In still other embodiments the vaccine contains at least one additional MTV component. In some embodiments the one additional MTV component is derived from at least one of: gp52, p14, syngp52, MTV polymerase, synMTV polymerase, MTV transcriptase, synMTV transcriptase, MTV surface, synMTV surface, MTV transmembrane anchoring, synMTV transmembrane anchoring, MTV-associated polysaccharide, and synMTV-associated polysaccharide. In another embodiment there is at least a second MTV component derived from a target species different from the first MTV component. In still other embodiments the vaccine may further contain MTV derived ENV and POL. In still other embodiments at least one of the ENV, POL and MTV transition proteins is synthetically derived. In another embodiment the vaccine may also contain a polysaccharide. In still other embodiments the vaccine may comprise additional antigens to form a combination polyvalent vaccine. In certain embodiments the additional antigens are derived from at least one of: an oncovirus, and a tumor-associated antigen.

In some embodiments the vaccine contains MTV polypeptides coupled to a carrier protein. In certain embodiments the MTV polypeptides are EP3, p14, and an MTV transition protein; and, the carrier protein is KLH. In further embodiments the MTV transition protein is p10 as exemplified in SEQ. ID: 2 or a homologous sequence with at least 90% identity thereof. In certain other embodiments the carrier protein is at least one selected from the group of KLH, Concholepas concholepas hemocyanin, BSA, Cationized BSA, and Ovalbumin. In certain other embodiments the vaccine also contains additional antigens to form a compound polyvalent vaccine.

Thus, by way of non-limiting example, an MTV vaccine comprising EP3, p10, and p14 bound to KLH along with any additional pharmaceutically acceptable compounds may be prophylactically provided to an MTV naïve patient. In response to the vaccine the patient immune system mounts a response resulting in long term immunity from subsequent MTV exposure.

Other embodiments of the current invention provide for pharmaceutical compositions comprising MTV DNA, RNA and/or proteins described herein. These compositions may further comprise a pharmaceutically acceptable excipient, carrier, or diluent and do not contain any biologically harmful substances. The pharmaceutical compositions of the present invention may be formulated by one having ordinary skill in the art. Suitable pharmaceutical formulations are described in Remington's Pharmaceutical Sciences which is a standard reference text in the field which is herein incorporated by reference.

The pharmaceutical compositions may further comprise coloring or stabilizing agents, osmotic agents, antibacterial agents, or any other substances as long as such substances do not interfere with the function of the composition. The pharmaceutical compositions of the instant invention, can, for example, be formulated as a solution, suspension, or emulsion in association with a pharmaceutically acceptable parenteral vehicle. Examples of such vehicles are water, saline, Ringer's solution, dextrose solution, and 5% human albumen. Liposomes may also be used. The vehicle may contain additives that maintain isotonicity (e.g., sodium chloride or mannitol) and chemical stability (e.g., buffers and preservatives). It should be appreciated that endotoxin contamination should be kept at a safe level, for example, less than 0.5 ng mg⁻¹ protein. Moreover, for human administration, preparations should meet sterility, pyrogenicity, general safety and purity standards as required by the United States Food and Drug Administration Office of Biological Standards. The formulations may be sterilized by commonly used techniques such as filtration.

The phrase “pharmaceutically acceptable” refers to substances and compositions which do not produce an adverse, allergic, or otherwise untoward reaction when administered to an animal, or a human, as appropriate. A substance which caused or produced any of these adverse effects would be classified as “biologically harmful” within the scope of the present invention. Pharmaceutically acceptable substances and compositions include, but are not limited to solvents, dispersion media, coatings, antibacterial and antifungal agents, isotonic and absorption delaying agents. Except where incompatible with the invention the use of any conventional ingredient is contemplated. Furthermore, supplementary active ingredients which serve some other pharmacologically expedient purpose can also be incorporated into the instant compositions without departing from the broader scope of the instant invention.

Another embodiment of the invention is a method of treating MTV where an MTV vaccine with an MTV transition protein is provided and administered to a patient. In some embodiments the vaccine is administered in conjunction with additional vaccines or other medical treatments as below described. In still other embodiments the vaccine is administered to at least one of a human, and a human associated animal. The vaccine may be administered as a preventative prophylactic to induce an immune response in a naïve host resulting in immunity to subsequent MTV exposure, or may be administered to an exposed patient for the purposes of bolstering an immune response to an extant viral presence. In such instances, either viral particle count or other measures of viral presence and/or activity are decreased as a result of vaccine administration.

Protease inhibitors are an example of one class of retroviral medications. As described above, MTV reproduction relies upon production and then cleavage of precursor proteins. In general, a protease inhibitor prevents cleavage of the viral precursor protein by inhibiting the action of the viral protease responsible for the cleavage. Thus, viral loads in a host are decreased as the virus fails to replicate. Accordingly, an embodiment of the invention results in the decrease of MTV loads in a patient accomplished through the administration of protease inhibitors. Example protease inhibitors can include at least one of: atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), and, darunavir/cobicistat (Prezcobix) either alone or in combination with another protease inhibitor. A method of treating a patient exposed to MTV may include: determining through an antibody or other test patient exposure status to MTV; then administering either a treatment dose or a maintenance dose of a protease inhibitor. In another embodiment, the protease inhibitor may be administered in conjunction with an MTV vaccine of the like described above thus decreasing viral load in a patient. In other embodiments, the patient may, instead, have a cancer diagnosis, or present other cancer-associated risk factors, without measurement of MTV levels or detection of MTV presence. The diagnosed cancer may, for example, be breast cancer or prostate cancer, or another viral-mediated cancer. In still other embodiments the patient may be analyzed for one or more risk factors (e.g., familial history, work history, past cancer status, etc.). Patients deemed above a given risk threshold may be deemed “high risk” and provided with antiretroviral medications as above described.

A treatment dosage is used as commonly understood in the art as one calculated to immediately or gradually (the terms “immediately” or “gradually” understood to be qualitative and relative, not quantitative) reduce a high viral load in a patient. In certain embodiments, patient exposure to MTV is determined through the use of an antibody titer test. In particular, the antibody test may detect antibodies to at least one of: gp52, p10, and p14. A maintenance dosage, as commonly understood, is one calculated to sustain a low or non-detectable limit of viral particles. An initial treatment dosage may be decreased to a maintenance dosage. Likewise, a treatment dosage with one or more primary protease inhibitors may be effected and then replaced with a maintenance dosage of one or more secondary protease inhibitors. In similar fashion, maintenance dosages may be increased or decreased as demanded by either viral load measurements or treatment protocols.

By way of non-limiting example, ritonavir may be provided to a patient as a maintenance dosage in the range of 50-440 mg per day, or atazanvir at 300-400 mg per day. In combination, ritonavir may be used with a range of 50-400 mg per day combined with another protease inhibitor at 100-400 mg per day. Protease inhibitors may also be used in combination with chemotherapy drugs to first treat a cancer (e.g., breast cancer or other viral-mediated cancer) and then used singly to prevent cancer recurrence. As used herein, dosage ranges explicitly contemplate every integer or part dosage amount (e.g., 1, 1.1, 1.2, 1.3, etc.) within the range including the initial and end amounts within the range. It is recognized that while ranges may list start and end points manufacturing tolerances and the like may result in products above or below range within a specified tolerance.

A method of treating MTV exposure in a patient can comprise: determining the MTV exposure status of a patient; and, administering a protease inhibitor to a patient with MTV exposure. In a further embodiment, the protease inhibitor may be administered in conjunction with an MTV vaccine. In still another embodiment the MTV vaccine may contain a transition protein. In still another embodiment the transition protein may be p10.

The method described above may further be modified wherein the administration of the protease inhibitor is performed at a first treatment dosage and a second maintenance dosage. In some embodiments an MTV vaccine may be administered during the treatment dosage administration. In still other embodiments, the MTV vaccine may be administered when switching to a maintenance dosage. In some embodiments the maintenance dosage is in the range of 50-200 mg of medication per day.

The method described above may be further modified wherein the protease inhibitor is administered in conjunction with chemotherapeutic agents in a cancerous patient. The protease inhibitor dosage may be altered from a treatment dosage to a maintenance dosage or switched to a second protease inhibitor at a maintenance dosage upon either cessation of chemotherapy treatments or remission of cancer. In certain cases, the cancer may be breast cancer, prostate cancer or another viral-mediated cancer.

In some embodiments of the method described above the protease inhibitor is at least one of: atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), and, darunavir/cobicistat (Prezcobix). In still other embodiments ritonavir is combined with at least one more protease inhibitor. In some embodiments the ritonavir is combined with at least one of: atazanavir (Reyataz), darunavir (Prezista), fosamprenavir (Lexiva), indinavir (Crixivan), lopinavir/ritonavir (Kaletra), nelfinavir (Viracept), ritonavir (Norvir), saquinavir (Invirase), tipranavir (Aptivus), atazanavir/cobicistat (Evotaz), and, darunavir/cobicistat (Prezcobix).

In some embodiments of the method above the protease inhibitor is ritonavier provided as a maintenance dosage in the range of 50-440 mg per day, or atazanvir at 300-400 mg per day.

In some embodiments, ritonavir may be used with a range of 50-400 mg per day and combined with another protease inhibitor at 100-400 mg per day.

In another embodiment of the invention a patient is first tested for the presence of MTV. Upon discovery of evidence of MTV exposure, the patient is then administered an antiretroviral medication. In some embodiments the antiretroviral medication is a protease inhibitor. In still other embodiments the protease inhibitor is administered at a first dosage level, corresponding to a treatment level, and then altered to a second or more dosage level corresponding to a maintenance level. In still other embodiments the test for MTV is an antibody test. In still other embodiments the antibody test relies on antibodies raised in response to at least one of: GP52, a transition protein, and p14. In still other embodiments the transition protein is p10.

For example, a patient with MTV exposure may first be treated with ritonavir at a dosage of 70 mg per day, concomitantly an MTV vaccine with an MTV transition protein is administered. The net effect results in an overall decrease in patient MTV viral particle loading and/or measured activity. Upon lowering of the MTV viral particle loading and/or measured activity, a second maintenance dosage of antiretroviral medications may be started. The net effect of the combined treatment is to halt the spread of an MTV viral-mediated cancer resulting in remission and/or the prevention of relapse.

In another embodiment, a patient with a cancer diagnosis is administered an antiretroviral medication. In some embodiments the antiretroviral medication is a protease inhibitor. In some embodiments the cancer diagnosis is for breast cancer. In still other embodiments the cancer diagnosis is for a viral-mediated cancer. In still other embodiments the protease inhibitor is administered at a first dosage level, corresponding to a treatment level, and then altered to a second or more dosage level corresponding to a second or more maintenance level. The method described above may further be modified wherein the administration of the protease inhibitor is performed at a first treatment dosage and a second maintenance dosage. In some embodiments an MTV vaccine may be administered during the treatment dosage administration. In still other embodiments, the MTV vaccine may be administered when switching to a maintenance dosage. In some embodiments the maintenance dosage is in the range of 50-200 mg of medication per day. In still other embodiments the MTV vaccine contains an MTV transition protein.

The effective dose and method of administration of a particular embodiment of the instant invention may vary based on the individual patient and stage of any present diseases (e.g., breast cancer, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by an individual physician in view of a patient to be treated. Dosage and administration are adjusted to provide sufficient levels of embodiments of the instant invention to maintain the desired effect (e.g., elimination or reduction of MTV particles or activity in a host). Additional factors that may be taken into account include the severity of any disease state, age, weight, and gender of the patient; diet, time and frequency of the administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks or more. Depending on half-life and clearance rate of the particular formulation, the pharmaceutical compositions of the instant invention may be administered once, twice, three, four, five, six, seven, eight, nine, ten or more times per day.

Normal dosage amounts may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of peptide agents described herein is one that provides sufficient peptide agent to attain a desirable effect, including stimulation of the immune system to produce antibodies, and/or disruption of the production of MTV viral particles. Accordingly, the dose of the peptide agent preferably produces a tissue or blood concentration of both about 1 to 800 μM. Preferable doses produces a tissue or blood concentration of greater than about 10 μM to about 500 μM. Preferable doses are, for example, the amount of peptide required to achieve a tissue or blood concentration or both of 10 μM, 15 μM, 20 μM, 25 μM, 30 μM, 35 μM, 40 PM, 45 μM, 50 μM, 55 μM, 60 μM, 65 μM, 70 μM, 75 μM, 80 μM, 85 μM, 90 μM, 95 μM, 100 PM, 110 μM, 120 μM, 130 μM, 140 μM, 150 μM, 160 μM, 170 μM, 180 μM, 190 μM, 200 PM, 220 μM, 240 μM, 250 μM, 260 μM, 280 μM, 300 μM, 320 μM, 340 μM, 360 μM, 380 μM, 400 PM, 420 μM, 440 μM, 460 μM, 480 μM, and 500 μM. Although doses that produce a tissue concentration greater than 800 μM are not necessarily preferred, they are envisioned and can be used with some embodiments of the present invention. A constant infusion of embodiments of the invention can be provided so as to maintain a stable concentration of the therapeutic agents (e.g., modified MTV peptides, MTV transition proteins, additional proteins or peptides co-administered, and other medicines such as retrovirals as herein described).

Protein N-myristoylation is a cotranslational lipidic modification specific to the alpha-amino group of an N-terminal glycine residue of many eukaryotic and viral proteins. The ubiquitous eukaryotic enzyme, N-myristoyltransferase (NMT), catalyzes the myristoylation process. Precisely, attachment of a myristoyl group increases specific protein-protein interactions leading to subcellular localization of myristoylated proteins with their signaling partners. The birth of the field of myristoylation, a little over three decades ago, has led to the understanding of the significance of protein myristoylation in regulating cellular signaling pathways in several biological processes especially in carcinogenesis and, more recently, immune function.

Typically, the myristate moiety participates in protein subcellular localization by facilitating protein-membrane interactions as well as protein-protein interactions. Myristoylated proteins are crucial components of a wide variety of functions, which include many signaling pathways, oncogenesis or viral replication. The attachment of myristic acid to the N-terminus is catalyzed by the ubiquitous eukaryotic enzyme, N-myristoyltransferase (NMT); a prosurvival protein, which uses myristoyl-coenzyme A (CoA) as a substrate. Although myristoylation typically occurs cotranslationally on newly synthesized polypeptides following cleavage of the initiator methionine by methionine aminopeptidase, there is also evidence that NMT-mediated posttranslational modification of proteins occurs after proteolytic cleavage of an N-terminal glycine residue.

Myristic acid is a hydrophobic moiety, and as the cell microenvironment is hydrophilic, the myristoylated protein is inserted into hydrophobic regions within the cell namely on: lipid rafts, the plasma membrane, endoplasmic reticulum (ER), Golgi apparatus, nuclear membrane, and mitochondria. Thus, depending on the subcellular localization of the myristoylated protein, it can regulate diverse cellular functions. Myristoylated proteins are adapted to performing biological functions in signal transduction, cellular transformation, and oncogenesis. Myristic acid is not an abundant fatty acid, it accounts for less than 1% of the total fatty acid in the cell, introducing another form of regulation.

Without subscribing to or limiting to a particular theory or practice, myristoylated proteins and/or peptide chains derived therefrom may further enhance the antigenic properties when used in combinations with further adjuvants. Hence, modified proteins and/or protein peptide chains may be myristoylated and used alone or in pharmaceutical compositions as described above to trigger or enhance an immune response leading to decreased or eliminated viral particle loads and/or activity. In particular, myristoylation may provide a means of anchoring an antigenic protein or peptide chain into cell membranes or carrier molecules enhancing their presentment to the immune system for an increased immune response resulting in either lasting immunity in a patient or decreased viral loading and/or activity.

There are often protein purification tags applied to peptide sequences to ease purification of synthetic proteins from a production system. For example, in immobilized metal ion chromatography (IMAC), a his-tag, or polyhistidine tag, is a string of histidine residues added at either the N or C terminus of a recombinant protein. There can be from four to ten residues in a string, although commonly there are six histidine residues a hexahistidine tag. Some recombinant proteins are engineered to have two or more hexahistidine tags.

In IMAC, transition metal ions are immobilized on a resin matrix using a chelating agent such as iminodiacetic acid. The most common ion for his-tag purification of a recombinant protein is Ni2+, though Co2+, Cu2+, and Zn2+ are also used. The his-tag has a high affinity for these metal ions and binds strongly to the IMAC column. Most other proteins in the lysate will not bind to the resin or bind only weakly. The use of a his-tag and IMAC can often provide relatively pure recombinant protein directly from a crude lysate.

Imidazole competes with the his-tag for binding to the metal-charged resin and thus is used for elution of the protein from an IMAC column. Typically, a low concentration of imidazole is added to both binding and wash buffers to interfere with the weak binding of other proteins and to elute any proteins that weakly bind. His-tagged protein is then eluted with a higher concentration of imidazole. Proteins with different numbers of poly histidine tags elute differently from nickel-affinity resin. For example, proteins with a single hexahistidine tag may elute from a Ni-NTA column at 75 mM of imidazole whereas proteins with two hexahistadine tags may require 100 mM imidazole solution to elute from the same column.

Some example alternatives to His-tag purification commonly include: HQ-tag (alternating histidine and glutamine); HN-tag (alternating histidine and asparagine), HAT-tag which is a peptide tag. Practitioners of ordinary skill in the art will readily recognize that any suitable purification tag may be added to embodiments without departing from the scope of the invention.

Embodiments of the present invention provide for isolated cDNA, DNA, RNA, and polypeptides derived from viral transition proteins including those from HMTV and MMTV and human associated animals. Accordingly an embodiment of the present invention provides for a recombinant cDNA plasmid (a vector) which comprises MTV DNA at least 90% identical to SEQ ID No: 2. In some embodiments, the plasmid vector further comprises a heterologous promoter operably linked to the MTV sequence (i.e., joined in the proper reading frame so as to be capable of producing functional MTV RNA and/or protein in vivo and in vitro). In one aspect of this embodiment of the invention the vector, which comprises the MTV DNA, is capable of episomal replication or chromosomal integration in at least one of the following cell types: bacterial cells, yeast cells, insect cells, avian cells, and mammalian cells (this list of cell types is representative and should not be considered exhaustive). In still other embodiments, the MTV DNA is modified to include an N-terminal glycine residue capable of myristoylation. In still other embodiments an artificial cDNA encoding a transition protein sequence, such as that of SEQ ID No: 2, comprises: a promoter sequence; a first position methionine; a second position glycine; a portion of or all of the sequence of a transition protein; one or more purification tags; a stop codon; and, the appropriate promoters and coding sequences for one or more additional genes for co-expression. In some embodiments the additional genes encoded include: myristoyl-CoA:protein N-myristoyl-transferase (NMT). In certain other expression systems, the additional genes may further include methionine aminopeptidase. Those of ordinary skill in the art without undue experimentation will recognize the need for sequence modifications adapted to their unique vector and/or protein expression systems without departing from the scope of the invention. Thus, those of skill in the art will recognize that one of the end products of this process is a myristoylated transition protein peptide chain. In some embodiments the myristoylated transition protein peptide chain is 90% or more homologous with the peptide encoded by SEQ ID No: 2. In still other embodiments the myristoylated transition protein peptide chain is 10 or more contiguous amino acids as found in SEQ ID No: 2 or an acceptably homologous sequence.

An example of an artificial cDNA sequence containing MTV protein p10 is provided at SEQ ID No: 3. In this example, the p10 sequence is altered to include an additional N-terminal glycine residue after an added initial methionine residue in order to enable myristoylation of the produced peptide. The sequence further includes a hexahistidine tag sequence with a 3× Gly linker. The sequence is left open to reflect the possibility of adding additional sequences. Those of ordinary skill in the art will be able to select appropriate promoter sequences for creation of final vector forms. In DNA/cDNA sequences example promotion sequences can include TATA box/Pribnow box sequences.

In certain embodiments the cDNA is incorporated into a vector, such as a plasmid, which is then subsequently expressed in the expression systems noted above for peptide/protein production. The peptide chains, with or without myristoylation are then purified and then incorporated into an acceptable pharmaceutical preparation as above disclosed. In certain embodiments a “linker” sequence (e.g., gly-gly-gly or gly-ser-gly) may be incorporated between the peptide chain and the his-tag. In subsequent purification steps the his-tag may then be removed.

In an example embodiment a purified transition protein peptide chain alone or in combination with a myristol group is combined into a pharmaceutical preparation as above described and administered to a patient with confirmed MTV exposure or, displaying MTV risk factors, or with a concomitant cancer diagnosis (viral-mediated or otherwise) with a resulting decrease in MTV viral load or activity.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The pharmacologically active compounds of this invention can be processed in accordance with conventional methods of galenic pharmacy to produce medicinal agents for administration to patients (e.g., mammals including humans). The peptides with, or without, modification can be incorporated into a pharmaceutical composition. Further, the manufacture of pharmaceuticals or therapeutic agents that deliver the peptides or a nucleic acid sequence encoding a peptide by several routes is an embodiment. By way of non-limiting example, DNA, RNA, and viral vectors having sequences that encoding a transition protein peptide with antigenic properties are contemplated. Nucleic acids encoding a desired transition protein peptide can be administered alone or in combination with a transition protein peptide resulting in lowered MTV viral particle counts or lowered measurements of MTV virus activity either via direct action or enhanced immune response.

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A composition, comprising:
a MTV transition protein; and
at least one adjuvant.

2. The composition of claim 1, wherein:
the MTV transition protein is protein p10 of SEQ. ID NO: 2; and
the at least one adjuvant is selected from the group of: alum, Complete Freund's Adjuvant, aluminum salt, and calcium salt.

3. The composition of claim 2, further comprising:
at least one MTV polypeptide selected from the group of: EP3 and p14;
wherein the p10 of SEQ. ID No: 2 and the at least one MTV polypeptide is coupled to a carrier protein.

4. The composition of claim 3, wherein:
the carrier protein is at least one selected from the group of: KLH, Concholepas concholepas hemocyanin, BSA, Cationized BSA, and Ovalbumin.

5. The composition of claim 1, further comprising:
MTV sourced ENV, and POL.

6. The composition of claim 1, further comprising:
additional antigens.

7. The composition of claim 6, wherein:
the additional antigens are sourced from at least one selected from the group of: Epstein-Barr Virus (EBV), human immunodeficiency virus (HIV), human papilloma virus (HPV), herpes simplex virus (HSV), and bovine leukemia virus (BLV).

8. The composition of claim 6, wherein:
the additional antigens are sourced from an RNA virus.

9. The composition of claim 1, further comprising:
at least one MTV polypeptide antigen sourced from at least one selected from the group of: gp52, p14, syngp52, MTV polymerase, synMTV polymerase, MTV transcriptase, synMTV transcriptase, MTV surface, synMTV surface, MTV transmembrane anchoring; and, synMTV transmembrane anchoring.

10. A method, comprising:
administering to a subject the composition of claim 1.

11. The method of claim 11, further comprising:
administering the composition of claim 1, in combination with at least one additional medication.

12. A vaccine comprising:
an MTV transition protein.

13. The vaccine of claim 12, wherein:
the MTV transition protein is p10.

14. The vaccine of claim 12, wherein:
the MTV transition protein is p10 of SEQ. ID NO. 2.

15. The vaccine of claim 12, further comprising:
additional antigens to form a combination vaccine.

16. The vaccine of claim 15, wherein:
the additional antigens are derived from at least one of: an oncovirus, and a tumor-associated antigen, to form a combination vaccine.

17. The vaccine of claim 12, further comprising:
at least one additional MTV component.

18. The vaccine of claim 12, further comprising:
MTV derived ENV and POL.

19. The vaccine of claim 18, wherein:
at least one of the ENV, POL, and MTV transition proteins is synthetically derived.

20. The vaccine of claim 12, further comprising:
additional antigens to form a combination polyvalent vaccine;
wherein the additional antigens are derived from at least one of: an oncovirus, and a tumor-associated antigen.
